(19) United States
(12) Patent Application Publication
(10) Pub. No.: US 2015/0106031 A1
(43) Pub. Date: Apr. 16, 2015

(54) CHARACTERIZATION OF CRUDE OIL BY NEAR INFRARED SPECTROSCOPY

(71) Applicants: Omer Refa Koseoglu, Dhahran (SA); Adnan Al-Hajji, Dammam (SA); Anaam Al-Shaikh Ali, Eastern Province (SA)

(72) Inventors: Omer Refa Koseoglu, Dhahran (SA); Adnan Al-Hajji, Dammam (SA); Anaam Al-Shaikh Ali, Eastern Province (SA)

(21) Appl. No.: 13/397,300

(22) Filed: Feb. 15, 2012

Related U.S. Application Data
(60) Provisional application No. 61/445,199, filed on Feb. 22, 2011.

(51) Int. Cl. G06F 19/00; G01N 21/359; G01N 21/3504; G01N 33/28; G01J 3/42

(52) U.S. Cl. CPC G01N 21/359; G01N 33/28; G01N 21/3504; G01N 2201/12
USPC 702/24; 702/23

(57) ABSTRACT

A system and a method for calculating the cetane number, pour point, cloud point and aniline point of a gas oil fraction of a crude oil from the density and near infrared spectroscopy of a sample of the crude oil.

### RELATED APPLICATION

This application claims the benefit of U.S. Provisional Patent Application No. 61/445,199 filed Feb. 22, 2011, the disclosure of which is hereby incorporated by reference.

### FIELD OF THE INVENTION

This invention relates to a method and process for the evaluation of samples of crude oil and its fractions by near infrared spectroscopy, avoiding the need to conduct crude oil assays.

### BACKGROUND OF THE INVENTION

Crude oil originates from the decomposition and transformation of aquatic, mainly marine, living organisms and/or land plants that became buried under successive layers of mud and silt some 15-500 million years ago. They are essentially very complex mixtures of many thousands of different hydrocarbons. Depending on the source, the oil predominantly contains various proportions of straight and branched-chain paraffins, cycloparaffins, and naphthenic, aromatic, and polynuclear aromatic hydrocarbons. These hydrocarbons can be gaseous, liquid, or solid under normal conditions of temperature and pressure, depending on the number and arrangement of carbon atoms in the molecules.

Crude oils vary widely in their physical and chemical properties from one geographical region to another and from field to field. Crude oils are usually classified into three groups according to the nature of the hydrocarbons they contain: paraffinic, naphthenic, asphaltic, and their mixtures. The differences are due to the different proportions of the various molecular types and sizes. One crude oil can contain mostly paraffins, another mostly naphthenes. Whether paraffinic or naphthenic, one can contain a large quantity of lighter hydrocarbons and be mobile or contain dissolved gases; another can consist mainly of heavier hydrocarbons and be highly viscous, with little or no dissolved gas. Crude oils can also include heteroatoms containing sulfur, nitrogen, nickel, vanadium and other elements in quantities that impact the refinery processing of the crude oil fractions. Light crude oils or condensates can contain sulfur in concentrations as low as 0.01 W %; in contrast, heavy crude oils can contain as much as 5-6 W %. Similarly, the nitrogen content of crude oils can range from 0.001-1.0 W %.

The nature of the crude oil governs, to a certain extent, the nature of the products that can be manufactured from it and their suitability for special applications. A naphthenic crude oil will be more suitable for the production of asphaltic bitumen, a paraffinic crude oil for wax. A naphthenic crude oil, and even more so an aromatic one, will yield lubricating oils with viscosities that are sensitive to temperature. However, with modern refining methods there is greater flexibility in the use of various crude oils to produce many desired type of products.

A crude oil assay is a traditional method of determining the nature of crude oils for benchmarking purposes. Crude oils are subjected to true boiling point (TBP) distillations and fractionations to provide different boiling point fractions. The crude oil distillations are carried out using the American Standard Testing Association (ASTM) Method D 2892. The common fractions and their nominal boiling points are given in Table 1.

TABLE 1

| Fraction | Boiling Point, ° C. |
|---|---|
| Methane | −161.5 |
| Ethane | −88.6 |
| Propane | −42.1 |
| Butanes | −6.0 |
| Light Naphtha | 36-90 |
| Mid Naphtha | 90-160 |
| Heavy Naphtha | 160-205 |
| Light gas Oil | 205-260 |
| Mid Gas Oil | 260-315 |
| Heavy gas Oil | 315-370 |
| Light Vacuum Gas Oil | 370-430 |
| Mid Vacuum Gas Oil | 430-480 |
| Heavy vacuum gas oil | 480-565 |
| Vacuum Residue | 565+ |

The yields, composition, physical and indicative properties of these crude oil fractions, where applicable, are then determined during the crude assay work-up calculations. Typical compositional and property information obtained from a crude oil assay is given in Table 2.

TABLE 2

| Property | Unit | Property Type | Fraction |
|---|---|---|---|
| Yield Weight and Volume % | W % | Yield | All |
| API Gravity | ° | Physical | All |
| Viscosity Kinematic @ 38° C. | ° | Physical | Fraction boiling >250° C. |
| Refractive Index @ 20° C. | Unitless | Physical | Fraction boiling <400° C. |
| Sulfur | W % | Composition | All |
| Mercaptan Sulfur, W % | W % | Composition | Fraction boiling <250° C. |
| Nickel | ppmw | Composition | Fraction boiling >400° C. |
| Nitrogen | ppmw | Composition | All |
| Flash Point, COC | ° C. | Indicative | All |
| Cloud Point | ° C. | Indicative | Fraction boiling >250° C. |
| Pour Point, (Upper) | ° C. | Indicative | Fraction boiling >250° C. |
| Freezing Point | ° C. | Indicative | Fraction boiling >250° C. |
| Microcarbon Residue | W % | Indicative | Fraction boiling >300° C. |
| Smoke Point, mm | mm | Indicative | Fraction boiling between |
| | | | 150-250 |
| Cetane Index | Unitless | Indicative | Fraction boiling between |
| | | | 150-400 |
| Aniline Point | ° C. | Indicative | Fraction boiling <520° C. |

Due to the number of distillation cuts and the number of analyses involved, the crude oil assay work-up is both costly and time consuming.

In a typical refinery, crude oil is first fractionated in the atmospheric distillation column to separate sour gas and light hydrocarbons, including methane, ethane, propane, butanes and hydrogen sulfide, naphtha (36°-180° C.), kerosene (180°-240° C.), gas oil (240°-370° C.) and atmospheric residue (>370° C.). The atmospheric residue from the atmospheric distillation column is either used as fuel oil or sent to a vacuum distillation unit, depending on the configuration of the refinery. The principal products obtained from vacuum distillation are vacuum gas oil, comprising hydrocarbons boiling in the range 370°-520° C., and vacuum residue, comprising hydrocarbons boiling above 520° C. The crude assay data help refiners to understand the general composition of the crude oil fractions and properties so that the fractions can be processed most efficiently and effectively in an appropriate refining unit. Indicative properties are used to determine the engine/fuel performance or usability or flow characteristic or composition. A summary of the indicative properties and their determination methods with description are given below.

Viscosity is a measure of the resistance of a fluid which is being deformed by either shear stress or tensile stress. Viscosity describes a fluid's internal resistance to flow and may be thought of as a measure of fluid friction. All real fluids (except superfluids) have some resistance to stress, but a fluid which has no resistance to shear stress is known as an ideal fluid or inviscid fluid. Viscosity of many petroleum fuels is important for the estimation of process units, optimum storage, handling, and operational conditions and determined by ASTM method D445.

The cetane number of diesel fuel oil, determined by the ASTM D613 method, provides a measure of the ignition quality of diesel fuel; as determined in a standard single cylinder test engine; which measures ignition delay compared to primary reference fuels. The higher the cetane number; the easier the high-speed; direct-injection engine will start; and the less white smoking and diesel knock after start-up are. The cetane number of a diesel fuel oil is determined by comparing its combustion characteristics in a test engine with those for blends of reference fuels of known cetane number under standard operating conditions. This is accomplished using the bracketing hand wheel procedure which varies the compression ratio (hand wheel reading) for the sample and each of the two bracketing reference fuels to obtain a specific ignition delay, thus permitting interpolation of cetane number in terms of hand wheel reading.

The cloud point, determined by the ASTM D2500 method, is the temperature at which a cloud of wax crystals appears when a lubricant or distillate fuel is cooled under standard conditions. Cloud point indicates the tendency of the material to plug filters or small orifices under cold weather conditions. The specimen is cooled at a specified rate and examined periodically. The temperature at which cloud is first observed at the bottom of the test jar is recorded as the cloud point. This test method covers only petroleum products and biodiesel fuels that are transparent in 40 mm thick layers, and with a cloud point below 49° C.

The pour point of petroleum products, determined by the ASTM D97 method, is an indicator of the ability of oil or distillate fuel to flow at cold operating temperatures. It is the lowest temperature at which the fluid will flow when cooled under prescribed conditions. After preliminary heating, the sample is cooled at a specified rate and examined at intervals of 3° C. for flow characteristics. The lowest temperature at which movement of the specimen is observed is recorded as the pour point.

The aniline point, determined by the ASTM D611 method, is the lowest temperature at which equal volumes of aniline and hydrocarbon fuel or lubricant base stock are completely miscible. A measure of the aromatic content of a hydrocarbon blend is used to predict the solvency of a base stock or the cetane number of a distillate fuel Specified volumes of aniline and sample, or aniline and sample plus n-heptane, are placed in a tube and mixed mechanically. The mixture is heated at a controlled rate until the two phases become miscible. The mixture is then cooled at a controlled rate and the temperature at which two phases separate is recorded as the aniline point or mixed aniline point.

To determine these properties of gas oil or naphtha fractions conventionally, these fractions have to be distilled off from the crude oil and then measured/determined using various analytical methods that are laborious, costly and time consuming.

Infrared energy is the electromagnetic energy of molecular vibration. The energy band is defined for convenience as the near infrared (0.78-2.50 microns), the infrared (or mid-infrared) 2.50-40.0 microns, and the far infrared (40.0-1000 microns). However, even though official standards, textbooks, and the scientific literature generally state that the NIR spectral region extends from 780-2500 nanometers (12821-4000 cm−1), a simple set of liquid phase hydrocarbon spectra demonstrates that the vibrational information characterized by the harmonic vibrations of the C—H stretch fundamental and their corresponding combination bands occurs from approximately 690-3000 nm. The predominant near-infrared spectral features include: the methyl C—H stretching vibrations, methylene C—H stretching vibrations, aromatic C—H stretching vibrations, and O—H stretching vibrations. Minor but still important spectral features include: methoxy C—H stretching, carbonyl associated C—H stretching; N—H from primary amides, secondary amides (both alkyl, and aryl group associations), N—H from primary, secondary, and tertiary amines, and N—H from amine salts.

Qualitative and quantitative near infrared (NIR) spectroscopic methods typically require the application of multivariate calibration algorithms and statistical methods (i.e. chemometrics) to model NIR spectral response to chemical or physical properties of the samples used for calibration. The NIR method relies on the spectra-structure correlations existing between a measured spectral response caused by the harmonics of the fundamental vibrations occurring at infrared frequencies. These harmonic vibrations occur at unique frequencies depending upon the quantity of absorber (analyte), type of absorbing molecules present within the sample, and the sample thickness. Quantitative methods are possible where changes in the response of the near infrared spectrometer are proportional to changes in the concentration of chemical components, or in the physical characteristics (scattering/absorptive properties) of samples undergoing analysis

Near infrared spectroscopy is used where multicomponent molecular vibrational analysis is required in the presence of interfering substances. The near infrared spectra consist of overtones and combination bands of the fundamental molecular absorptions found in the mid infrared region. Near infrared spectra consist of generally overlapping vibrational bands that may appear non-specific and poorly resolved. The use of chemometric mathematical data processing and multiple harmonics can be used to calibrate for qualitative of quantitative analysis despite these apparent spectroscopic limitations. Traditional near infrared spectroscopy has been most often used for analysis of lignin polymers (2270 nm), paraffins and long alkane chain polymers (2310 nm), glucose based polymers such as cellulose (2336 nm), amino acid polymers as proteins (2180 nm), carbohydrates (2100 nm), and moisture (1440 and 1940 nm). When analyzing synthetic and natural materials NIR spectroscopy has shown unprecedented industrial success in multiple applications. The basic uses of near infrared spectroscopy have been for process control, quality assessment, identification of raw materials and process byproducts, and chemical quantitative analysis of complex mixtures.

Note that a near infrared spectrum consists in the convolution of the measuring instrument function with the unique optical characteristics of the sample being measured (i.e. the sample is an active optical element of the spectrometer). The reference values are those chemical or physical parameters to be predicted using the NIR spectroscopic measurements. A spectrum may, or may not, contain information related to the sample chemistry measured using any specific reference method. Spectra-structure correlation provides a basis for the establishment of a known cause and effect relationship between instrument response and reference (analyte) data, in order to provide a more scientific basis for multivariate-based near infrared spectroscopy. When performing multivariate calibrations, analytically valid calibration models require a relationship between X (the instrument response data or spectrum), and Y (the reference data). The use of probability alone tells us only if X and Y ‘appear’ to be related. If no cause-effect relationship exists between spectra-structure correlation and reference values the model will have no true predictive importance. Thus, knowledge of cause and effect creates a basis for scientific decision-making.

Factors affecting the integrity of the teaching samples used to calibrate spectrophotometers for individual NIR applications include the variations in sample chemistry, the physical condition of samples, and the measurement conditions. Teaching Sets must represent several sample ‘spaces’ to include: compositional space, instrument space, and measurement condition (sample handling and presentation) space. Interpretive spectroscopy is a key intellectual process in approaching NIR measurements if one is to achieve an analytical understanding of these measurements.

Near-infrared (NIR) spectroscopy has been employed for the characterization of products derived from petroleum, such as gasoline and diesel fuel, with considerable success. The intrinsic capacity of the NIR spectrum to obtain information on the different types of C—H bonds as well as other chemical bonds of interest (such as S—H and N—H) has been proved to be valuable in the prediction of quality parameters such as octane number, ethanol content, MTBE (methyl tert-butyl ether) content, distillation points, Reid vapor pressure and aromatic and saturated contents in gasoline. The information present in the NIR spectrum can be successfully applied to the prediction of quality parameters for diesel fuel such as cetane number, distillation points, density, viscosity and sulfur content.

Any new rapid, direct method to help better understand the crude oil composition and properties from the analysis of whole crude oil will save producers, marketers, refiners and/or other crude oil users substantial expense, effort and time. Therefore, a need exists for an improved system and method for determining the properties of crude oil fractions from different sources and classifying the crude oil fractions based on their boiling point characteristics and/or properties.

### SUMMARY OF THE INVENTION

The above objects and further advantages are provided by the present invention which broadly comprehends a system and a method for determining the indicative properties of a hydrocarbon sample. In accordance with the invention, indicative properties (i.e., cetane number, pour point, cloud point and aniline point) of gas oil fraction in crude oils are predicted by density and direct near infrared spectroscopy measurement of crude oils. The correlations also provide information about the gas oil properties without fractionation/distillation (crude oil assays) and will help producers, refiners, and marketers to benchmark the oil quality and, as a result, valuate the oils without performing the customary extensive and time-consuming crude oil assays.

### BRIEF DESCRIPTION OF THE DRAWINGS

Further advantages and features of the present invention will become apparent from the following detailed description of the invention when considered with reference to the accompanying drawings, in which:

FIG. 1 is a graphic plot of typical near infrared spectroscopy data for three types of crude oil;

FIG. 2 is a process flow diagram of steps carried out to characterize the API gravity of a crude oil sample, using the system and method of the present invention; and

FIG. 3 is a block diagram of a component of a system for implementing the invention, according to one preferred embodiment of the present invention.

### DETAILED DESCRIPTION OF INVENTION

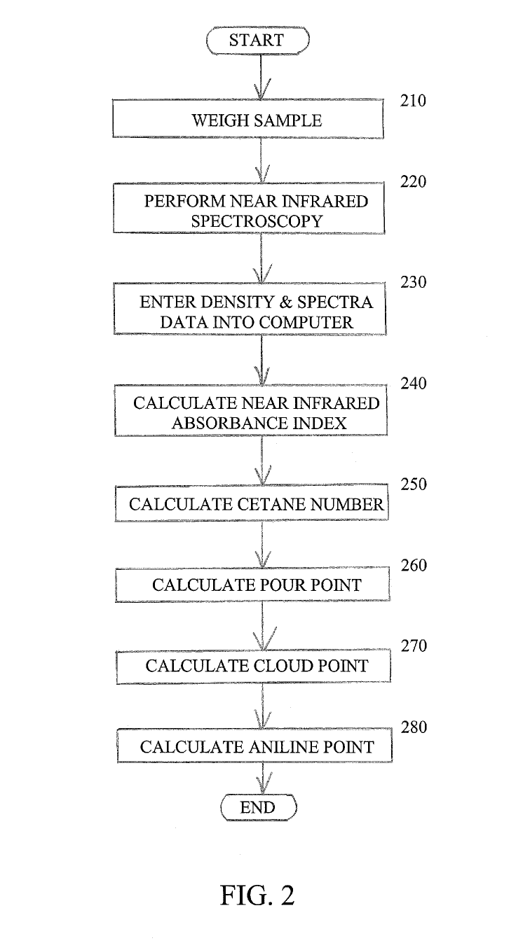

FIG. 2 shows a process flowchart of steps that occur in one embodiment of the invention. In step 210, a crude oil sample is weighed. In step 220, crude oils were analyzed by near infrared spectroscopy in accordance with the instructions of the equipment manufacturer. No dilution or special preparation is required.

Equation (1) shows a near infrared absorbance index (NIRA):

$$NIRA = {\sum }_{i = 4 , 000}^{12 , 821} ( {Absorbance}_{( i )} ) / 10 , 000 ; \qquad ( 1 )$$

where:

Absorbance=absorbance value of the crude oil solution for peaks detected at wavenumbers over the range 4,000 cm⁻¹ to 12,821 cm⁻¹.

In step 230, the density and spectra data are entered into a computer. In step 240, the NIRA is calculated.

The indicative properties (i.e., the cetane number, pour point, cloud point and aniline point) of the gas oil fraction boiling in the range 180-370° C. can be predicted from the density of whole crude oil and the near infrared absorbance index (NIRA) of crude oil. That is,

Indicative Property=f(density_{crude oil},NIRA_{crude}) (2);

Equations (3) through (6) are detailed examples of this relationship, respectively showing the cetane number, pour point, cloud point and aniline point that can be predicted from the density and near infrared spectroscopy of crude oils.

Cetane Number(CET)=K_{CET}+X1_{CET}*DEN+X2_{CET}*DEN²+X3_{CET}*DEN³+X4_{CET}*NIRA+X5_{CET}*NIRA²+X6_{CET}*NIRA³+X7_{CET}*DEN*NIRA (3);

Pour Point(PP)=K_{PP}+X1_{PP}*DEN+X2_{PP}*DEN²+X3_{PP}*DEN³+X4_{PP}*NIRA+X5_{PP}*NIRA²+X6_{PP}*NIRA³+X7_{PP}*DEN*NIRA (4);

Cloud Point(CP)=K_{CP}+X1_{CP}*DEN+X2_{CP}*DEN²+X3_{CP}*DEN³+X4_{CP}*NIRA+X5_{CP}*NIRA²+X6_{CP}*NIRA³+X7_{CP}*DEN*NIRA (5);

Aniline Point(AP)=K_{AP}+X1_{AP}*DEN+X2_{AP}*DEN²+X3_{AP}*DEN³+X4_{AP}*NIRA+X5_{AP}*NIRA²+X6_{AP}*NIRA³+X7_{AP}*DEN*NIRA (6);

where:

DEN=density of the crude oil sample;

NIRA=near infrared absorbance (derived from near infrared spectra);

and K_{CET}, X1_{CET}-X7_{CET}, K_{PP}, X1_{PP}-X7_{PP}, K_{CP}, X1_{CP}-X7_{CP}, K_{AP}, and X1_{AP}-X7_{AP} are constants that were developed using linear regression techniques, and which are given in Table 3.

In step 250, the cetane number is calculated. In step 260, the pour point is calculated. In step 270, the cloud point is calculated. In step 280, the aniline point is calculated.

TABLE 3

| | Cetane Number | | Cloud | Aniline |
|---|---|---|---|---|
| Property | (CET) | Pour Point (PP) | Point (CP) | Point (AP) |
| K | −181844.7 | −1373741.5 | −567845.2 | 760795.9 |
| X1 | 576360.4 | 4551764.5 | 1890384.7 | −2548841.4 |
| X2 | −593268.7 | −4984097.0 | −2082457.0 | 2831541.0 |
| X3 | 196815.2 | 1802114.2 | 758773.6 | −1042818.6 |
| X4 | −14874.9 | −43934.6 | −15153.0 | 14832.5 |
| X5 | 1230.5 | 5414.9 | 2411.3 | −2461.4 |
| X6 | −916.9 | −3397.2 | −1430.5 | 1412.8 |
| X7 | 16450.1 | 47134.2 | 15879.7 | −15424.7 |

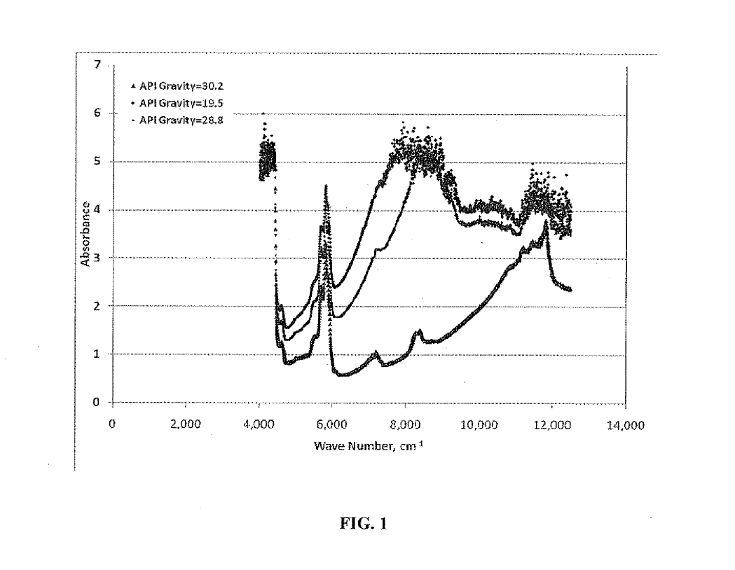

The following example is provided. A sample of Arabian medium crude with a density of 0.8828 Kg/1 was analyzed by near infrared spectroscopy. The spectra data is presented in Table 4 and is shown in FIG. 1 as the sample with an API gravity of 28.8°. The near infrared spectroscopy index (NIRA) is calculated by summing the absorbances of the detected peaks and then dividing by 10,000, with the value in the example calculated as 0.7576.

Applying equation 3 and the constants from Table 3,

Cetane Number(CET)=K_{CET}+X1_{CET}*DEN+X2_{CET}*DEN²+X3_{CET}*DEN³+X4_{CET}*NIRA+X5_{CET}*NIRA²+X6_{CET}*NIRA³+X7_{CET}*DEN*NIRA

=(−181844.7)+(576360.4)(0.8828)+(−593268.7)(0.8828)²+(196815.2)(0.8828)³+(−14874.9)(0.7576)+(1230.5)(0.7576)²+(−916.9)(0.7576)³+(16450.1)(0.8828)(0.7576)=59

Applying equation 4 and the constants from Table 3,

Pour Point(PP)=K_{PP}+X1_{PP}*DEN+X2_{PP}*DEN²+X3_{PP}*DEN³+X4_{PP}*NIRA+X5_{PP}*NIRA²+X6_{PP}*NIRA³+X7_{PP}*DEN*NIRA

=(−1373741.5)+(4551764.5)(0.8828)+(−4984097.0)(0.8828)²+(1802114.2)(0.8828)³+(−43934.6)(0.7576)+(5414.9)(0.7576)²+(−3397.2)(0.7576)³+(47134.2)(0.8828)(0.7576)=−9° C.

Applying equation 5 and the constants from Table 3,

Cloud Point(CP)=K_{CP}+X1_{CP}*DEN+X2_{CP}*DEN²+X3_{CP}*DEN³+X4_{CP}*NIRA+X5_{CP}*NIRA²+X6_{CP}*NIRA³+X7_{CP}*DEN*NIRA

=(−567845.2)+(1890384.7)(0.8828)+(−2082457.0)(0.8828)²+(758773.6)(0.8888)³+(−15153.0)(0.7576)+(2411.3)(0.7576)²+(−1430.5)(0.7576)³+(15879.7)(0.8828)(0.7576)=−10° C.

Applying equation 6 and the constants from Table 3,

Aniline Point(AP)=K_{AP}+X1_{AP}*DEN+X2_{AP}*DEN²+X3_{AP}*DEN³+X4_{AP}*NIRA+X5_{AP}*NIRA²+X6_{AP}*NIRA³+X7_{AP}*DEN*NIRA

=(760795.9)+(−2548841.4)(0.8828)+(2831541.0)(0.8828)²+(−1042818.6)(0.8828)³+(14832.5)(0.7576)+(−2461.4)(0.7576)²+(1412.8)(0.7576)³+(−15424.7)(0.8828)(0.7576)=66° C.

The method is applicable for naturally occurring hydrocarbons derived from crude oils, bitumens, heavy oils, shale oils and from refinery process units including hydrotreating, hydroprocessing, fluid catalytic cracking, coking, and visbreaking or coal liquefaction.

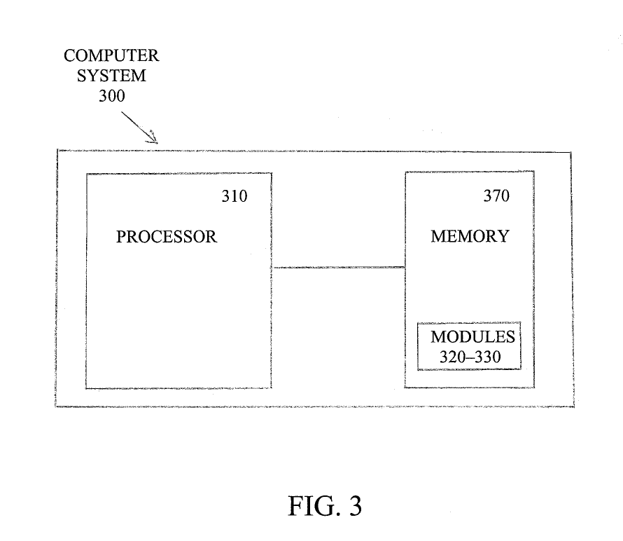

FIG. 3 illustrates an embodiment of the present invention, implemented in a computer system 300, with a number of modules. Computer system 300 includes a processor 310, and a memory unit 370. Memory unit 370 stores software program modules and associated data, and in particular stores a near infrared absorbance index (NIRA) calculation module 320, a cetane number calculation module 322, a pour point calculation module 324, a cloud point calculation module 326, and an aniline point calculation module 330.

The system and method of the present invention have been described above and with reference to the attached figure; however, modifications will be apparent to those of ordinary skill in the art and the scope of protection for the invention is to be defined by the claims that follow.

TABLE 4

| cm⁻¹ | Absorb. | cm⁻¹ | Absorb. | cm⁻¹ | Absorb. | cm⁻¹ | Absorb. | cm⁻¹ | Absorb. |
|---|---|---|---|---|---|---|---|---|---|
| 12493 | 3.61 | 10792 | 3.64 | 9091 | 4.37 | 7390 | 3.21 | 5689 | 3.18 |
| 12489 | 3.56 | 10788 | 3.65 | 9087 | 4.28 | 7386 | 3.21 | 5685 | 3.2 |
| 12485 | 3.52 | 10784 | 3.65 | 9084 | 4.25 | 7383 | 3.21 | 5682 | 3.23 |
| 12482 | 3.48 | 10781 | 3.62 | 9080 | 4.3 | 7379 | 3.21 | 5678 | 3.24 |
| 12478 | 3.51 | 10777 | 3.61 | 9076 | 4.42 | 7375 | 3.2 | 5674 | 3.24 |
| 12474 | 3.56 | 10773 | 3.62 | 9072 | 4.48 | 7371 | 3.2 | 5670 | 3.23 |
| 12470 | 3.57 | 10769 | 3.64 | 9068 | 4.41 | 7367 | 3.2 | 5666 | 3.19 |
| 12466 | 3.56 | 10765 | 3.64 | 9064 | 4.36 | 7363 | 3.2 | 5662 | 3.15 |
| 12462 | 3.58 | 10761 | 3.67 | 9060 | 4.36 | 7359 | 3.21 | 5658 | 3.09 |
| 12458 | 3.6 | 10757 | 3.7 | 9057 | 4.37 | 7356 | 3.21 | 5655 | 3.03 |
| 12455 | 3.6 | 10754 | 3.68 | 9053 | 4.35 | 7352 | 3.21 | 5651 | 2.96 |
| 12451 | 3.65 | 10750 | 3.61 | 9049 | 4.36 | 7348 | 3.21 | 5647 | 2.9 |
| 12447 | 3.74 | 10746 | 3.58 | 9045 | 4.38 | 7344 | 3.21 | 5643 | 2.84 |
| 12443 | 3.72 | 10742 | 3.6 | 9041 | 4.45 | 7340 | 3.2 | 5639 | 2.78 |
| 12439 | 3.68 | 10738 | 3.62 | 9037 | 4.52 | 7336 | 3.2 | 5635 | 2.72 |
| 12435 | 3.67 | 10734 | 3.64 | 9033 | 4.51 | 7332 | 3.19 | 5631 | 2.66 |
| 12431 | 3.63 | 10730 | 3.66 | 9030 | 4.42 | 7329 | 3.19 | 5628 | 2.6 |
| 12428 | 3.6 | 10727 | 3.68 | 9026 | 4.34 | 7325 | 3.18 | 5624 | 2.54 |
| 12424 | 3.57 | 10723 | 3.68 | 9022 | 4.32 | 7321 | 3.18 | 5620 | 2.49 |
| 12420 | 3.51 | 10719 | 3.67 | 9018 | 4.34 | 7317 | 3.18 | 5616 | 2.44 |
| 12416 | 3.5 | 10715 | 3.66 | 9014 | 4.37 | 7313 | 3.18 | 5612 | 2.39 |
| 12412 | 3.57 | 10711 | 3.67 | 9010 | 4.41 | 7309 | 3.18 | 5608 | 2.35 |
| 12408 | 3.59 | 10707 | 3.66 | 9006 | 4.45 | 7305 | 3.17 | 5604 | 2.31 |
| 12404 | 3.54 | 10703 | 3.65 | 9003 | 4.44 | 7302 | 3.17 | 5601 | 2.28 |
| 12401 | 3.57 | 10700 | 3.64 | 8999 | 4.43 | 7298 | 3.17 | 5597 | 2.26 |
| 12397 | 3.68 | 10696 | 3.62 | 8995 | 4.49 | 7294 | 3.17 | 5593 | 2.24 |
| 12393 | 3.7 | 10692 | 3.6 | 8991 | 4.55 | 7290 | 3.17 | 5589 | 2.22 |
| 12389 | 3.63 | 10688 | 3.6 | 8987 | 4.57 | 7286 | 3.18 | 5585 | 2.21 |
| 12385 | 3.58 | 10684 | 3.61 | 8983 | 4.57 | 7282 | 3.18 | 5581 | 2.2 |
| 12381 | 3.58 | 10680 | 3.64 | 8979 | 4.58 | 7278 | 3.18 | 5577 | 2.2 |
| 12377 | 3.57 | 10676 | 3.67 | 8976 | 4.58 | 7275 | 3.18 | 5574 | 2.19 |
| 12374 | 3.57 | 10673 | 3.68 | 8972 | 4.55 | 7271 | 3.18 | 5570 | 2.18 |
| 12370 | 3.57 | 10669 | 3.68 | 8968 | 4.48 | 7267 | 3.18 | 5566 | 2.18 |
| 12366 | 3.56 | 10665 | 3.67 | 8964 | 4.46 | 7263 | 3.18 | 5562 | 2.17 |
| 12362 | 3.55 | 10661 | 3.65 | 8960 | 4.49 | 7259 | 3.19 | 5558 | 2.17 |
| 12358 | 3.56 | 10657 | 3.64 | 8956 | 4.52 | 7255 | 3.19 | 5554 | 2.16 |
| 12354 | 3.56 | 10653 | 3.65 | 8952 | 4.53 | 7251 | 3.19 | 5550 | 2.15 |
| 12350 | 3.54 | 10649 | 3.67 | 8949 | 4.55 | 7248 | 3.19 | 5547 | 2.15 |
| 12347 | 3.51 | 10646 | 3.69 | 8945 | 4.59 | 7244 | 3.19 | 5543 | 2.14 |
| 12343 | 3.5 | 10642 | 3.69 | 8941 | 4.62 | 7240 | 3.19 | 5539 | 2.13 |
| 12339 | 3.53 | 10638 | 3.69 | 8937 | 4.65 | 7236 | 3.19 | 5535 | 2.13 |
| 12335 | 3.6 | 10634 | 3.68 | 8933 | 4.69 | 7232 | 3.19 | 5531 | 2.12 |
| 12331 | 3.64 | 10630 | 3.66 | 8929 | 4.73 | 7228 | 3.19 | 5527 | 2.12 |
| 12327 | 3.59 | 10626 | 3.64 | 8925 | 4.77 | 7224 | 3.19 | 5523 | 2.12 |
| 12323 | 3.54 | 10622 | 3.66 | 8922 | 4.86 | 7221 | 3.19 | 5520 | 2.12 |
| 12320 | 3.57 | 10619 | 3.68 | 8918 | 4.84 | 7217 | 3.19 | 5516 | 2.12 |
| 12316 | 3.68 | 10615 | 3.66 | 8914 | 4.75 | 7213 | 3.19 | 5512 | 2.12 |
| 12312 | 3.68 | 10611 | 3.62 | 8910 | 4.82 | 7209 | 3.2 | 5508 | 2.12 |
| 12308 | 3.57 | 10607 | 3.61 | 8906 | 4.98 | 7205 | 3.2 | 5504 | 2.12 |
| 12304 | 3.5 | 10603 | 3.64 | 8902 | 5.07 | 7201 | 3.2 | 5500 | 2.12 |
| 12300 | 3.48 | 10599 | 3.7 | 8898 | 4.85 | 7197 | 3.2 | 5496 | 2.12 |
| 12296 | 3.5 | 10595 | 3.75 | 8895 | 4.72 | 7194 | 3.2 | 5493 | 2.11 |
| 12293 | 3.6 | 10592 | 3.75 | 8891 | 4.68 | 7190 | 3.21 | 5489 | 2.11 |
| 12289 | 3.66 | 10588 | 3.72 | 8887 | 4.79 | 7186 | 3.21 | 5485 | 2.1 |
| 12285 | 3.64 | 10584 | 3.71 | 8883 | 5.05 | 7182 | 3.2 | 5481 | 2.1 |
| 12281 | 3.69 | 10580 | 3.71 | 8879 | 4.87 | 7178 | 3.19 | 5477 | 2.09 |
| 12277 | 3.7 | 10576 | 3.71 | 8875 | 4.98 | 7174 | 3.18 | 5473 | 2.08 |
| 12273 | 3.62 | 10572 | 3.71 | 8871 | 5.11 | 7170 | 3.17 | 5469 | 2.08 |
| 12269 | 3.56 | 10568 | 3.68 | 8868 | 5.22 | 7167 | 3.16 | 5466 | 2.07 |
| 12266 | 3.53 | 10565 | 3.64 | 8864 | 5.1 | 7163 | 3.14 | 5462 | 2.06 |
| 12262 | 3.56 | 10561 | 3.63 | 8860 | 4.98 | 7159 | 3.12 | 5458 | 2.04 |
| 12258 | 3.63 | 10557 | 3.64 | 8856 | 4.93 | 7155 | 3.11 | 5454 | 2.03 |
| 12254 | 3.74 | 10553 | 3.65 | 8852 | 4.99 | 7151 | 3.1 | 5450 | 2.02 |
| 12250 | 3.88 | 10549 | 3.66 | 8848 | 5.02 | 7147 | 3.08 | 5446 | 2.01 |
| 12246 | 3.79 | 10545 | 3.67 | 8844 | 4.72 | 7143 | 3.07 | 5442 | 1.99 |
| 12242 | 3.7 | 10541 | 3.69 | 8841 | 4.65 | 7140 | 3.06 | 5439 | 1.98 |
| 12239 | 3.64 | 10538 | 3.69 | 8837 | 4.74 | 7136 | 3.05 | 5435 | 1.97 |
| 12235 | 3.55 | 10534 | 3.69 | 8833 | 4.88 | 7132 | 3.04 | 5431 | 1.95 |
| 12231 | 3.48 | 10530 | 3.7 | 8829 | 4.92 | 7128 | 3.04 | 5427 | 1.94 |
| 12227 | 3.47 | 10526 | 3.71 | 8825 | 4.94 | 7124 | 3.03 | 5423 | 1.92 |
| 12223 | 3.46 | 10522 | 3.71 | 8821 | 4.85 | 7120 | 3.03 | 5419 | 1.91 |
| 12219 | 3.45 | 10518 | 3.7 | 8817 | 4.73 | 7116 | 3.02 | 5415 | 1.89 |
| 12215 | 3.44 | 10514 | 3.68 | 8814 | 4.81 | 7113 | 3.02 | 5412 | 1.88 |
| 12212 | 3.42 | 10511 | 3.67 | 8810 | 5.14 | 7109 | 3.02 | 5408 | 1.86 |
| 12208 | 3.47 | 10507 | 3.68 | 8806 | 5.04 | 7105 | 3.02 | 5404 | 1.84 |
| 12204 | 3.61 | 10503 | 3.71 | 8802 | 4.98 | 7101 | 3.02 | 5400 | 1.83 |
| 12200 | 3.73 | 10499 | 3.75 | 8798 | 4.94 | 7097 | 3.02 | 5396 | 1.81 |
| 12196 | 3.67 | 10495 | 3.75 | 8794 | 5.18 | 7093 | 3.02 | 5392 | 1.8 |
| 12192 | 3.6 | 10491 | 3.72 | 8790 | 5.24 | 7089 | 3.02 | 5388 | 1.78 |
| 12188 | 3.6 | 10487 | 3.7 | 8787 | 5.23 | 7086 | 3.02 | 5385 | 1.77 |
| 12185 | 3.61 | 10484 | 3.72 | 8783 | 5.04 | 7082 | 3.02 | 5381 | 1.76 |
| 12181 | 3.59 | 10480 | 3.75 | 8779 | 4.91 | 7078 | 3.02 | 5377 | 1.74 |
| 12177 | 3.61 | 10476 | 3.74 | 8775 | 4.95 | 7074 | 3.01 | 5373 | 1.73 |
| 12173 | 3.66 | 10472 | 3.71 | 8771 | 5.01 | 7070 | 3 | 5369 | 1.72 |
| 12169 | 3.65 | 10468 | 3.72 | 8767 | 4.97 | 7066 | 2.99 | 5365 | 1.72 |
| 12165 | 3.62 | 10464 | 3.73 | 8763 | 4.89 | 7062 | 2.97 | 5361 | 1.71 |
| 12161 | 3.62 | 10460 | 3.72 | 8760 | 4.83 | 7059 | 2.96 | 5358 | 1.7 |
| 12158 | 3.59 | 10457 | 3.72 | 8756 | 4.84 | 7055 | 2.95 | 5354 | 1.7 |
| 12154 | 3.55 | 10453 | 3.71 | 8752 | 4.89 | 7051 | 2.93 | 5350 | 1.69 |
| 12150 | 3.56 | 10449 | 3.7 | 8748 | 4.8 | 7047 | 2.92 | 5346 | 1.69 |
| 12146 | 3.6 | 10445 | 3.72 | 8744 | 4.82 | 7043 | 2.91 | 5342 | 1.68 |
| 12142 | 3.59 | 10441 | 3.75 | 8740 | 5.06 | 7039 | 2.9 | 5338 | 1.68 |
| 12138 | 3.57 | 10437 | 3.76 | 8736 | 5.1 | 7035 | 2.89 | 5334 | 1.68 |
| 12134 | 3.62 | 10433 | 3.73 | 8733 | 4.99 | 7032 | 2.88 | 5331 | 1.68 |
| 12131 | 3.7 | 10430 | 3.72 | 8729 | 4.98 | 7028 | 2.88 | 5327 | 1.67 |
| 12127 | 3.73 | 10426 | 3.7 | 8725 | 4.95 | 7024 | 2.87 | 5323 | 1.67 |
| 12123 | 3.7 | 10422 | 3.67 | 8721 | 4.87 | 7020 | 2.86 | 5319 | 1.67 |
| 12119 | 3.62 | 10418 | 3.68 | 8717 | 4.93 | 7016 | 2.86 | 5315 | 1.67 |
| 12115 | 3.57 | 10414 | 3.7 | 8713 | 4.93 | 7012 | 2.85 | 5311 | 1.66 |
| 12111 | 3.59 | 10410 | 3.74 | 8709 | 4.84 | 7008 | 2.84 | 5307 | 1.66 |
| 12107 | 3.64 | 10406 | 3.78 | 8706 | 5.04 | 7005 | 2.84 | 5304 | 1.66 |
| 12104 | 3.62 | 10403 | 3.77 | 8702 | 5.14 | 7001 | 2.83 | 5300 | 1.65 |
| 12100 | 3.57 | 10399 | 3.75 | 8698 | 5.07 | 6997 | 2.83 | 5296 | 1.65 |
| 12096 | 3.55 | 10395 | 3.73 | 8694 | 5.2 | 6993 | 2.83 | 5292 | 1.65 |
| 12092 | 3.54 | 10391 | 3.74 | 8690 | 5.17 | 6989 | 2.82 | 5288 | 1.65 |
| 12088 | 3.53 | 10387 | 3.74 | 8686 | 5.13 | 6985 | 2.82 | 5284 | 1.64 |
| 12084 | 3.52 | 10383 | 3.74 | 8682 | 5.54 | 6981 | 2.81 | 5280 | 1.64 |
| 12080 | 3.53 | 10379 | 3.72 | 8679 | 5.32 | 6978 | 2.81 | 5277 | 1.64 |
| 12077 | 3.6 | 10376 | 3.72 | 8675 | 5.11 | 6974 | 2.8 | 5273 | 1.64 |
| 12073 | 3.72 | 10372 | 3.75 | 8671 | 5.29 | 6970 | 2.79 | 5269 | 1.63 |
| 12069 | 3.76 | 10368 | 3.76 | 8667 | 5.02 | 6966 | 2.78 | 5265 | 1.63 |
| 12065 | 3.74 | 10364 | 3.77 | 8663 | 4.72 | 6962 | 2.78 | 5261 | 1.62 |
| 12061 | 3.72 | 10360 | 3.77 | 8659 | 4.72 | 6958 | 2.77 | 5257 | 1.62 |
| 12057 | 3.71 | 10356 | 3.76 | 8655 | 4.85 | 6954 | 2.76 | 5253 | 1.62 |
| 12053 | 3.72 | 10352 | 3.75 | 8652 | 4.95 | 6951 | 2.75 | 5250 | 1.61 |
| 12050 | 3.77 | 10349 | 3.75 | 8648 | 4.98 | 6947 | 2.74 | 5246 | 1.61 |
| 12046 | 3.78 | 10345 | 3.77 | 8644 | 4.99 | 6943 | 2.74 | 5242 | 1.61 |
| 12042 | 3.7 | 10341 | 3.77 | 8640 | 4.99 | 6939 | 2.73 | 5238 | 1.6 |
| 12038 | 3.62 | 10337 | 3.75 | 8636 | 4.97 | 6935 | 2.72 | 5234 | 1.6 |
| 12034 | 3.64 | 10333 | 3.72 | 8632 | 4.96 | 6931 | 2.71 | 5230 | 1.6 |
| 12030 | 3.8 | 10329 | 3.68 | 8628 | 4.93 | 6927 | 2.7 | 5226 | 1.6 |
| 12026 | 3.98 | 10325 | 3.68 | 8625 | 4.88 | 6924 | 2.7 | 5223 | 1.59 |
| 12023 | 3.98 | 10322 | 3.72 | 8621 | 4.98 | 6920 | 2.69 | 5219 | 1.59 |
| 12019 | 3.87 | 10318 | 3.77 | 8617 | 5.51 | 6916 | 2.68 | 5215 | 1.59 |
| 12015 | 3.77 | 10314 | 3.79 | 8613 | 5.23 | 6912 | 2.67 | 5211 | 1.59 |
| 12011 | 3.7 | 10310 | 3.75 | 8609 | 5.12 | 6908 | 2.67 | 5207 | 1.58 |
| 12007 | 3.71 | 10306 | 3.73 | 8605 | 5.18 | 6904 | 2.66 | 5203 | 1.58 |
| 12003 | 3.79 | 10302 | 3.76 | 8601 | 5.05 | 6900 | 2.65 | 5199 | 1.58 |
| 11999 | 3.89 | 10298 | 3.79 | 8598 | 4.87 | 6897 | 2.64 | 5196 | 1.57 |
| 11996 | 3.88 | 10295 | 3.76 | 8594 | 4.85 | 6893 | 2.63 | 5192 | 1.57 |
| 11992 | 3.76 | 10291 | 3.74 | 8590 | 4.85 | 6889 | 2.63 | 5188 | 1.57 |
| 11988 | 3.66 | 10287 | 3.76 | 8586 | 5.01 | 6885 | 2.62 | 5184 | 1.56 |
| 11984 | 3.63 | 10283 | 3.79 | 8582 | 5.73 | 6881 | 2.61 | 5180 | 1.56 |
| 11980 | 3.65 | 10279 | 3.79 | 8578 | 4.99 | 6877 | 2.6 | 5176 | 1.56 |
| 11976 | 3.7 | 10275 | 3.78 | 8574 | 4.93 | 6873 | 2.59 | 5172 | 1.55 |
| 11972 | 3.78 | 10271 | 3.78 | 8571 | 4.96 | 6870 | 2.59 | 5169 | 1.55 |
| 11969 | 3.83 | 10268 | 3.78 | 8567 | 4.96 | 6866 | 2.58 | 5165 | 1.55 |
| 11965 | 3.78 | 10264 | 3.77 | 8563 | 5.04 | 6862 | 2.57 | 5161 | 1.54 |
| 11961 | 3.69 | 10260 | 3.76 | 8559 | 5.13 | 6858 | 2.57 | 5157 | 1.54 |
| 11957 | 3.64 | 10256 | 3.75 | 8555 | 5.1 | 6854 | 2.56 | 5153 | 1.54 |
| 11953 | 3.64 | 10252 | 3.74 | 8551 | 4.93 | 6850 | 2.56 | 5149 | 1.53 |
| 11949 | 3.69 | 10248 | 3.76 | 8547 | 4.8 | 6846 | 2.55 | 5145 | 1.53 |
| 11945 | 3.74 | 10244 | 3.79 | 8544 | 4.75 | 6843 | 2.54 | 5142 | 1.53 |
| 11942 | 3.73 | 10241 | 3.81 | 8540 | 4.76 | 6839 | 2.54 | 5138 | 1.53 |
| 11938 | 3.73 | 10237 | 3.77 | 8536 | 4.87 | 6835 | 2.53 | 5134 | 1.52 |
| 11934 | 3.8 | 10233 | 3.72 | 8532 | 5 | 6831 | 2.53 | 5130 | 1.52 |
| 11930 | 3.87 | 10229 | 3.7 | 8528 | 4.92 | 6827 | 2.52 | 5126 | 1.52 |
| 11926 | 3.89 | 10225 | 3.73 | 8524 | 4.93 | 6823 | 2.52 | 5122 | 1.52 |
| 11922 | 3.9 | 10221 | 3.77 | 8520 | 5.1 | 6819 | 2.51 | 5118 | 1.51 |
| 11918 | 3.95 | 10217 | 3.79 | 8517 | 5.32 | 6816 | 2.51 | 5115 | 1.51 |
| 11915 | 4.06 | 10214 | 3.76 | 8513 | 5.09 | 6812 | 2.5 | 5111 | 1.51 |
| 11911 | 4.15 | 10210 | 3.73 | 8509 | 4.82 | 6808 | 2.49 | 5107 | 1.51 |
| 11907 | 4.06 | 10206 | 3.73 | 8505 | 4.77 | 6804 | 2.49 | 5103 | 1.51 |
| 11903 | 3.94 | 10202 | 3.76 | 8501 | 4.84 | 6800 | 2.48 | 5099 | 1.51 |
| 11899 | 3.88 | 10198 | 3.77 | 8497 | 4.93 | 6796 | 2.48 | 5095 | 1.5 |
| 11895 | 3.9 | 10194 | 3.77 | 8493 | 4.81 | 6792 | 2.47 | 5091 | 1.5 |
| 11891 | 3.92 | 10190 | 3.78 | 8490 | 4.71 | 6789 | 2.46 | 5088 | 1.5 |
| 11888 | 3.92 | 10187 | 3.78 | 8486 | 4.74 | 6785 | 2.45 | 5084 | 1.5 |
| 11884 | 4.02 | 10183 | 3.78 | 8482 | 4.77 | 6781 | 2.45 | 5080 | 1.5 |
| 11880 | 4.12 | 10179 | 3.76 | 8478 | 4.77 | 6777 | 2.44 | 5076 | 1.49 |
| 11876 | 4.15 | 10175 | 3.75 | 8474 | 4.96 | 6773 | 2.44 | 5072 | 1.49 |
| 11872 | 4.16 | 10171 | 3.75 | 8470 | 5.11 | 6769 | 2.43 | 5068 | 1.49 |
| 11868 | 4.13 | 10167 | 3.78 | 8466 | 5.21 | 6765 | 2.42 | 5064 | 1.49 |
| 11864 | 4.03 | 10163 | 3.78 | 8463 | 5.07 | 6762 | 2.42 | 5061 | 1.48 |
| 11861 | 4.03 | 10160 | 3.75 | 8459 | 5.16 | 6758 | 2.41 | 5057 | 1.48 |
| 11857 | 4.28 | 10156 | 3.72 | 8455 | 5.11 | 6754 | 2.41 | 5053 | 1.48 |
| 11853 | 4.47 | 10152 | 3.73 | 8451 | 4.99 | 6750 | 2.4 | 5049 | 1.48 |
| 11849 | 4.29 | 10148 | 3.77 | 8447 | 4.92 | 6746 | 2.39 | 5045 | 1.48 |
| 11845 | 4.1 | 10144 | 3.8 | 8443 | 4.86 | 6742 | 2.39 | 5041 | 1.48 |
| 11841 | 4.16 | 10140 | 3.78 | 8439 | 4.93 | 6738 | 2.38 | 5037 | 1.48 |
| 11837 | 4.25 | 10136 | 3.76 | 8436 | 5.09 | 6735 | 2.38 | 5034 | 1.48 |
| 11834 | 4 | 10133 | 3.75 | 8432 | 5.09 | 6731 | 2.37 | 5030 | 1.47 |
| 11830 | 3.96 | 10129 | 3.74 | 8428 | 4.88 | 6727 | 2.37 | 5026 | 1.47 |
| 11826 | 4.01 | 10125 | 3.73 | 8424 | 4.85 | 6723 | 2.36 | 5022 | 1.47 |
| 11822 | 4.01 | 10121 | 3.71 | 8420 | 4.85 | 6719 | 2.36 | 5018 | 1.47 |
| 11818 | 4.01 | 10117 | 3.69 | 8416 | 4.85 | 6715 | 2.35 | 5014 | 1.47 |
| 11814 | 4.01 | 10113 | 3.69 | 8412 | 4.89 | 6711 | 2.34 | 5010 | 1.47 |
| 11810 | 4.05 | 10109 | 3.72 | 8409 | 5.09 | 6708 | 2.34 | 5007 | 1.47 |
| 11807 | 4.12 | 10106 | 3.73 | 8405 | 5.13 | 6704 | 2.33 | 5003 | 1.47 |
| 11803 | 4.17 | 10102 | 3.73 | 8401 | 5.06 | 6700 | 2.33 | 4999 | 1.47 |
| 11799 | 4.24 | 10098 | 3.74 | 8397 | 5.13 | 6696 | 2.32 | 4995 | 1.47 |
| 11795 | 4.37 | 10094 | 3.77 | 8393 | 5.04 | 6692 | 2.32 | 4991 | 1.47 |
| 11791 | 4.18 | 10090 | 3.77 | 8389 | 4.97 | 6688 | 2.31 | 4987 | 1.47 |
| 11787 | 4.09 | 10086 | 3.73 | 8385 | 5.11 | 6684 | 2.31 | 4983 | 1.47 |
| 11783 | 4.3 | 10082 | 3.71 | 8382 | 5.1 | 6681 | 2.3 | 4980 | 1.47 |
| 11780 | 4.27 | 10079 | 3.73 | 8378 | 5.06 | 6677 | 2.3 | 4976 | 1.47 |
| 11776 | 4.19 | 10075 | 3.75 | 8374 | 5.15 | 6673 | 2.29 | 4972 | 1.47 |
| 11772 | 4.12 | 10071 | 3.75 | 8370 | 5.08 | 6669 | 2.28 | 4968 | 1.46 |
| 11768 | 4.18 | 10067 | 3.73 | 8366 | 4.98 | 6665 | 2.28 | 4964 | 1.46 |
| 11764 | 4.13 | 10063 | 3.7 | 8362 | 4.88 | 6661 | 2.27 | 4960 | 1.45 |
| 11760 | 4.04 | 10059 | 3.71 | 8358 | 4.8 | 6657 | 2.27 | 4956 | 1.45 |
| 11756 | 4.05 | 10055 | 3.73 | 8355 | 4.81 | 6654 | 2.26 | 4953 | 1.44 |
| 11753 | 4.05 | 10052 | 3.76 | 8351 | 4.97 | 6650 | 2.26 | 4949 | 1.44 |
| 11749 | 4.15 | 10048 | 3.76 | 8347 | 5.11 | 6646 | 2.25 | 4945 | 1.43 |
| 11745 | 4.25 | 10044 | 3.76 | 8343 | 5.15 | 6642 | 2.25 | 4941 | 1.43 |
| 11741 | 4.26 | 10040 | 3.77 | 8339 | 5.36 | 6638 | 2.24 | 4937 | 1.42 |
| 11737 | 4.15 | 10036 | 3.78 | 8335 | 5.12 | 6634 | 2.23 | 4933 | 1.42 |
| 11733 | 4.17 | 10032 | 3.78 | 8331 | 5.03 | 6630 | 2.23 | 4929 | 1.41 |
| 11729 | 4.32 | 10028 | 3.78 | 8328 | 5.05 | 6627 | 2.22 | 4926 | 1.41 |
| 11726 | 4.35 | 10025 | 3.79 | 8324 | 5.1 | 6623 | 2.22 | 4922 | 1.4 |
| 11722 | 4.32 | 10021 | 3.81 | 8320 | 5.05 | 6619 | 2.21 | 4918 | 1.4 |
| 11718 | 4.21 | 10017 | 3.82 | 8316 | 5.14 | 6615 | 2.21 | 4914 | 1.39 |
| 11714 | 4.03 | 10013 | 3.8 | 8312 | 5.12 | 6611 | 2.2 | 4910 | 1.39 |
| 11710 | 4.08 | 10009 | 3.79 | 8308 | 5.05 | 6607 | 2.2 | 4906 | 1.39 |
| 11706 | 4.12 | 10005 | 3.82 | 8304 | 5.2 | 6603 | 2.19 | 4902 | 1.38 |
| 11702 | 4.02 | 10002 | 3.88 | 8301 | 4.99 | 6600 | 2.19 | 4899 | 1.38 |
| 11699 | 3.96 | 9998 | 3.89 | 8297 | 4.94 | 6596 | 2.18 | 4895 | 1.38 |
| 11695 | 4.12 | 9994 | 3.86 | 8293 | 5.27 | 6592 | 2.18 | 4891 | 1.38 |
| 11691 | 4.32 | 9990 | 3.83 | 8289 | 5.18 | 6588 | 2.17 | 4887 | 1.37 |
| 11687 | 4.4 | 9986 | 3.8 | 8285 | 5.06 | 6584 | 2.17 | 4883 | 1.37 |
| 11683 | 4.23 | 9982 | 3.77 | 8281 | 5.11 | 6580 | 2.16 | 4879 | 1.37 |
| 11679 | 4.22 | 9978 | 3.75 | 8277 | 5.06 | 6576 | 2.16 | 4875 | 1.37 |
| 11675 | 4.29 | 9975 | 3.74 | 8274 | 4.98 | 6573 | 2.15 | 4872 | 1.36 |
| 11672 | 4.46 | 9971 | 3.75 | 8270 | 4.96 | 6569 | 2.15 | 4868 | 1.36 |
| 11668 | 4.58 | 9967 | 3.77 | 8266 | 4.97 | 6565 | 2.15 | 4864 | 1.35 |
| 11664 | 4.41 | 9963 | 3.77 | 8262 | 4.99 | 6561 | 2.14 | 4860 | 1.35 |
| 11660 | 4.19 | 9959 | 3.76 | 8258 | 5.01 | 6557 | 2.14 | 4856 | 1.35 |
| 11656 | 4.11 | 9955 | 3.75 | 8254 | 5.07 | 6553 | 2.13 | 4852 | 1.34 |
| 11652 | 4.1 | 9951 | 3.75 | 8250 | 5.24 | 6549 | 2.13 | 4848 | 1.34 |
| 11648 | 4.15 | 9948 | 3.76 | 8247 | 5.24 | 6546 | 2.12 | 4845 | 1.33 |
| 11645 | 4.27 | 9944 | 3.79 | 8243 | 5.14 | 6542 | 2.12 | 4841 | 1.33 |
| 11641 | 4.2 | 9940 | 3.8 | 8239 | 5.21 | 6538 | 2.11 | 4837 | 1.33 |
| 11637 | 4.02 | 9936 | 3.8 | 8235 | 4.98 | 6534 | 2.11 | 4833 | 1.32 |
| 11633 | 4.08 | 9932 | 3.78 | 8231 | 4.82 | 6530 | 2.11 | 4829 | 1.32 |
| 11629 | 4.14 | 9928 | 3.77 | 8227 | 4.8 | 6526 | 2.1 | 4825 | 1.32 |
| 11625 | 4.07 | 9924 | 3.78 | 8223 | 4.8 | 6522 | 2.1 | 4821 | 1.32 |
| 11621 | 4.05 | 9921 | 3.78 | 8220 | 4.73 | 6519 | 2.09 | 4818 | 1.32 |
| 11618 | 4.11 | 9917 | 3.77 | 8216 | 4.69 | 6515 | 2.09 | 4814 | 1.31 |
| 11614 | 4.11 | 9913 | 3.78 | 8212 | 4.8 | 6511 | 2.08 | 4810 | 1.31 |
| 11610 | 4.11 | 9909 | 3.78 | 8208 | 4.87 | 6507 | 2.08 | 4806 | 1.31 |
| 11606 | 4.07 | 9905 | 3.77 | 8204 | 4.87 | 6503 | 2.07 | 4802 | 1.31 |
| 11602 | 4.11 | 9901 | 3.77 | 8200 | 4.84 | 6499 | 2.07 | 4798 | 1.31 |
| 11598 | 4.21 | 9897 | 3.78 | 8196 | 4.78 | 6495 | 2.07 | 4794 | 1.31 |
| 11594 | 4.15 | 9894 | 3.78 | 8193 | 4.75 | 6492 | 2.06 | 4791 | 1.31 |
| 11591 | 4.05 | 9890 | 3.76 | 8189 | 4.77 | 6488 | 2.06 | 4787 | 1.31 |
| 11587 | 3.99 | 9886 | 3.74 | 8185 | 4.84 | 6484 | 2.05 | 4783 | 1.31 |
| 11583 | 3.98 | 9882 | 3.73 | 8181 | 4.83 | 6480 | 2.05 | 4779 | 1.31 |
| 11579 | 4.05 | 9878 | 3.74 | 8177 | 4.67 | 6476 | 2.04 | 4775 | 1.31 |
| 11575 | 4.15 | 9874 | 3.77 | 8173 | 4.57 | 6472 | 2.04 | 4771 | 1.31 |
| 11571 | 4.21 | 9870 | 3.78 | 8169 | 4.56 | 6468 | 2.03 | 4767 | 1.31 |
| 11567 | 4.21 | 9867 | 3.76 | 8166 | 4.65 | 6465 | 2.03 | 4764 | 1.31 |
| 11564 | 4.4 | 9863 | 3.76 | 8162 | 4.74 | 6461 | 2.02 | 4760 | 1.31 |
| 11560 | 4.54 | 9859 | 3.77 | 8158 | 4.73 | 6457 | 2.02 | 4756 | 1.31 |
| 11556 | 4.37 | 9855 | 3.77 | 8154 | 4.73 | 6453 | 2.01 | 4752 | 1.31 |
| 11552 | 4.18 | 9851 | 3.74 | 8150 | 4.77 | 6449 | 2.01 | 4748 | 1.3 |
| 11548 | 4.18 | 9847 | 3.73 | 8146 | 4.82 | 6445 | 2 | 4744 | 1.3 |
| 11544 | 4.2 | 9843 | 3.74 | 8142 | 4.84 | 6441 | 2 | 4740 | 1.3 |
| 11540 | 4.16 | 9840 | 3.75 | 8139 | 4.79 | 6438 | 2 | 4737 | 1.3 |
| 11537 | 4.16 | 9836 | 3.74 | 8135 | 4.68 | 6434 | 1.99 | 4733 | 1.3 |
| 11533 | 4.14 | 9832 | 3.72 | 8131 | 4.57 | 6430 | 1.99 | 4729 | 1.3 |
| 11529 | 4.09 | 9828 | 3.73 | 8127 | 4.54 | 6426 | 1.98 | 4725 | 1.3 |
| 11525 | 4.05 | 9824 | 3.75 | 8123 | 4.55 | 6422 | 1.98 | 4721 | 1.3 |
| 11521 | 3.99 | 9820 | 3.75 | 8119 | 4.56 | 6418 | 1.97 | 4717 | 1.31 |
| 11517 | 3.94 | 9816 | 3.73 | 8115 | 4.59 | 6414 | 1.97 | 4713 | 1.31 |
| 11513 | 3.98 | 9813 | 3.72 | 8112 | 4.58 | 6411 | 1.97 | 4710 | 1.31 |
| 11510 | 4.03 | 9809 | 3.73 | 8108 | 4.57 | 6407 | 1.96 | 4706 | 1.32 |
| 11506 | 4.03 | 9805 | 3.74 | 8104 | 4.57 | 6403 | 1.96 | 4702 | 1.33 |
| 11502 | 4.04 | 9801 | 3.75 | 8100 | 4.54 | 6399 | 1.96 | 4698 | 1.34 |
| 11498 | 4.1 | 9797 | 3.75 | 8096 | 4.49 | 6395 | 1.95 | 4694 | 1.35 |
| 11494 | 4.21 | 9793 | 3.73 | 8092 | 4.45 | 6391 | 1.95 | 4690 | 1.36 |
| 11490 | 4.33 | 9789 | 3.74 | 8088 | 4.43 | 6387 | 1.94 | 4686 | 1.37 |
| 11486 | 4.28 | 9786 | 3.75 | 8085 | 4.44 | 6384 | 1.94 | 4683 | 1.39 |
| 11483 | 4.42 | 9782 | 3.76 | 8081 | 4.44 | 6380 | 1.94 | 4679 | 1.41 |
| 11479 | 4.27 | 9778 | 3.75 | 8077 | 4.46 | 6376 | 1.93 | 4675 | 1.44 |
| 11475 | 4.21 | 9774 | 3.73 | 8073 | 4.45 | 6372 | 1.93 | 4671 | 1.46 |
| 11471 | 4.1 | 9770 | 3.71 | 8069 | 4.39 | 6368 | 1.92 | 4667 | 1.48 |
| 11467 | 4.12 | 9766 | 3.7 | 8065 | 4.35 | 6364 | 1.92 | 4663 | 1.5 |
| 11463 | 4.13 | 9762 | 3.7 | 8061 | 4.35 | 6360 | 1.92 | 4659 | 1.51 |
| 11459 | 4.07 | 9759 | 3.71 | 8058 | 4.37 | 6357 | 1.91 | 4656 | 1.53 |
| 11456 | 4.16 | 9755 | 3.72 | 8054 | 4.4 | 6353 | 1.91 | 4652 | 1.54 |
| 11452 | 4.15 | 9751 | 3.72 | 8050 | 4.44 | 6349 | 1.91 | 4648 | 1.56 |
| 11448 | 4.07 | 9747 | 3.73 | 8046 | 4.44 | 6345 | 1.9 | 4644 | 1.57 |
| 11444 | 4.09 | 9743 | 3.73 | 8042 | 4.38 | 6341 | 1.9 | 4640 | 1.59 |
| 11440 | 4.18 | 9739 | 3.73 | 8038 | 4.36 | 6337 | 1.9 | 4636 | 1.6 |
| 11436 | 4.28 | 9735 | 3.72 | 8034 | 4.39 | 6333 | 1.89 | 4632 | 1.61 |
| 11432 | 4.2 | 9732 | 3.72 | 8031 | 4.38 | 6330 | 1.89 | 4629 | 1.63 |
| 11429 | 4.1 | 9728 | 3.7 | 8027 | 4.35 | 6326 | 1.89 | 4625 | 1.64 |
| 11425 | 4.22 | 9724 | 3.69 | 8023 | 4.33 | 6322 | 1.88 | 4621 | 1.66 |
| 11421 | 4.38 | 9720 | 3.69 | 8019 | 4.3 | 6318 | 1.88 | 4617 | 1.67 |
| 11417 | 4.25 | 9716 | 3.7 | 8015 | 4.26 | 6314 | 1.88 | 4613 | 1.68 |
| 11413 | 4.15 | 9712 | 3.72 | 8011 | 4.25 | 6310 | 1.87 | 4609 | 1.69 |
| 11409 | 4.05 | 9708 | 3.74 | 8007 | 4.27 | 6306 | 1.87 | 4605 | 1.7 |
| 11405 | 4.01 | 9705 | 3.74 | 8004 | 4.29 | 6303 | 1.87 | 4602 | 1.7 |
| 11402 | 4.11 | 9701 | 3.73 | 8000 | 4.27 | 6299 | 1.86 | 4598 | 1.7 |
| 11398 | 4.2 | 9697 | 3.72 | 7996 | 4.25 | 6295 | 1.86 | 4594 | 1.7 |
| 11394 | 4.26 | 9693 | 3.72 | 7992 | 4.23 | 6291 | 1.86 | 4590 | 1.69 |
| 11390 | 4.3 | 9689 | 3.72 | 7988 | 4.25 | 6287 | 1.85 | 4586 | 1.68 |
| 11386 | 4.11 | 9685 | 3.72 | 7984 | 4.26 | 6283 | 1.85 | 4582 | 1.67 |
| 11382 | 4.01 | 9681 | 3.72 | 7980 | 4.26 | 6279 | 1.85 | 4578 | 1.67 |
| 11378 | 4.05 | 9678 | 3.71 | 7977 | 4.24 | 6276 | 1.84 | 4575 | 1.66 |
| 11375 | 4.04 | 9674 | 3.71 | 7973 | 4.2 | 6272 | 1.84 | 4571 | 1.65 |
| 11371 | 4.02 | 9670 | 3.72 | 7969 | 4.18 | 6268 | 1.84 | 4567 | 1.64 |
| 11367 | 3.97 | 9666 | 3.72 | 7965 | 4.18 | 6264 | 1.84 | 4563 | 1.64 |
| 11363 | 3.93 | 9662 | 3.71 | 7961 | 4.19 | 6260 | 1.83 | 4559 | 1.63 |
| 11359 | 3.93 | 9658 | 3.7 | 7957 | 4.19 | 6256 | 1.83 | 4555 | 1.63 |
| 11355 | 3.99 | 9654 | 3.7 | 7953 | 4.17 | 6252 | 1.83 | 4551 | 1.63 |
| 11351 | 4.09 | 9651 | 3.71 | 7950 | 4.15 | 6249 | 1.82 | 4548 | 1.64 |
| 11348 | 4.18 | 9647 | 3.71 | 7946 | 4.15 | 6245 | 1.82 | 4544 | 1.64 |
| 11344 | 4.19 | 9643 | 3.69 | 7942 | 4.16 | 6241 | 1.82 | 4540 | 1.65 |
| 11340 | 4.16 | 9639 | 3.67 | 7938 | 4.18 | 6237 | 1.82 | 4536 | 1.65 |
| 11336 | 4.16 | 9635 | 3.67 | 7934 | 4.18 | 6233 | 1.81 | 4532 | 1.66 |
| 11332 | 4.07 | 9631 | 3.7 | 7930 | 4.16 | 6229 | 1.81 | 4528 | 1.67 |
| 11328 | 3.96 | 9627 | 3.73 | 7926 | 4.13 | 6225 | 1.81 | 4524 | 1.68 |
| 11324 | 3.94 | 9624 | 3.72 | 7923 | 4.1 | 6222 | 1.81 | 4521 | 1.69 |
| 11321 | 3.95 | 9620 | 3.7 | 7919 | 4.07 | 6218 | 1.8 | 4517 | 1.7 |
| 11317 | 4.01 | 9616 | 3.68 | 7915 | 4.07 | 6214 | 1.8 | 4513 | 1.71 |
| 11313 | 4.07 | 9612 | 3.68 | 7911 | 4.09 | 6210 | 1.8 | 4509 | 1.73 |
| 11309 | 4 | 9608 | 3.68 | 7907 | 4.09 | 6206 | 1.8 | 4505 | 1.74 |
| 11305 | 3.89 | 9604 | 3.7 | 7903 | 4.07 | 6202 | 1.79 | 4501 | 1.76 |
| 11301 | 3.84 | 9600 | 3.72 | 7899 | 4.05 | 6198 | 1.79 | 4497 | 1.78 |
| 11297 | 3.88 | 9597 | 3.72 | 7896 | 4.05 | 6195 | 1.79 | 4494 | 1.8 |
| 11294 | 3.97 | 9593 | 3.71 | 7892 | 4.06 | 6191 | 1.79 | 4490 | 1.83 |
| 11290 | 4.03 | 9589 | 3.7 | 7888 | 4.05 | 6187 | 1.79 | 4486 | 1.85 |
| 11286 | 3.99 | 9585 | 3.71 | 7884 | 4.03 | 6183 | 1.79 | 4482 | 1.88 |
| 11282 | 3.89 | 9581 | 3.71 | 7880 | 4.02 | 6179 | 1.79 | 4478 | 1.92 |
| 11278 | 3.86 | 9577 | 3.7 | 7876 | 4.01 | 6175 | 1.78 | 4474 | 1.96 |
| 11274 | 3.87 | 9573 | 3.7 | 7872 | 3.99 | 6171 | 1.78 | 4470 | 2 |
| 11270 | 3.85 | 9570 | 3.72 | 7869 | 3.97 | 6168 | 1.78 | 4467 | 2.05 |
| 11267 | 3.83 | 9566 | 3.74 | 7865 | 3.97 | 6164 | 1.78 | 4463 | 2.11 |
| 11263 | 3.85 | 9562 | 3.72 | 7861 | 3.97 | 6160 | 1.78 | 4459 | 2.17 |
| 11259 | 3.85 | 9558 | 3.71 | 7857 | 3.96 | 6156 | 1.78 | 4455 | 2.25 |
| 11255 | 3.84 | 9554 | 3.72 | 7853 | 3.96 | 6152 | 1.78 | 4451 | 2.35 |
| 11251 | 3.82 | 9550 | 3.74 | 7849 | 3.96 | 6148 | 1.78 | 4447 | 2.46 |
| 11247 | 3.82 | 9546 | 3.74 | 7845 | 3.96 | 6144 | 1.79 | 4443 | 2.59 |
| 11243 | 3.82 | 9543 | 3.74 | 7842 | 3.95 | 6141 | 1.79 | 4440 | 2.76 |
| 11240 | 3.81 | 9539 | 3.74 | 7838 | 3.95 | 6137 | 1.79 | 4436 | 2.95 |
| 11236 | 3.78 | 9535 | 3.74 | 7834 | 3.94 | 6133 | 1.79 | 4432 | 3.19 |
| 11232 | 3.75 | 9531 | 3.76 | 7830 | 3.92 | 6129 | 1.79 | 4428 | 3.47 |
| 11228 | 3.75 | 9527 | 3.77 | 7826 | 3.91 | 6125 | 1.79 | 4424 | 3.79 |
| 11224 | 3.83 | 9523 | 3.77 | 7822 | 3.91 | 6121 | 1.79 | 4420 | 4.12 |
| 11220 | 3.95 | 9519 | 3.77 | 7818 | 3.9 | 6117 | 1.79 | 4416 | 4.45 |
| 11216 | 4 | 9516 | 3.73 | 7815 | 3.9 | 6114 | 1.79 | 4413 | 4.82 |
| 11213 | 3.93 | 9512 | 3.71 | 7811 | 3.89 | 6110 | 1.79 | 4409 | 5.13 |
| 11209 | 3.82 | 9508 | 3.71 | 7807 | 3.88 | 6106 | 1.79 | 4405 | 5.19 |
| 11205 | 3.79 | 9504 | 3.73 | 7803 | 3.88 | 6102 | 1.79 | 4401 | 5.15 |
| 11201 | 3.87 | 9500 | 3.76 | 7799 | 3.88 | 6098 | 1.79 | 4397 | 5.1 |
| 11197 | 3.95 | 9496 | 3.78 | 7795 | 3.88 | 6094 | 1.79 | 4393 | 5.05 |
| 11193 | 3.89 | 9492 | 3.78 | 7791 | 3.88 | 6090 | 1.79 | 4389 | 5.05 |
| 11189 | 3.79 | 9489 | 3.77 | 7788 | 3.87 | 6087 | 1.79 | 4386 | 5.09 |
| 11186 | 3.72 | 9485 | 3.75 | 7784 | 3.86 | 6083 | 1.79 | 4382 | 5.2 |
| 11182 | 3.74 | 9481 | 3.73 | 7780 | 3.84 | 6079 | 1.79 | 4378 | 5.34 |
| 11178 | 3.79 | 9477 | 3.73 | 7776 | 3.84 | 6075 | 1.79 | 4374 | 5.39 |
| 11174 | 3.81 | 9473 | 3.75 | 7772 | 3.83 | 6071 | 1.78 | 4370 | 5.2 |
| 11170 | 3.8 | 9469 | 3.77 | 7768 | 3.82 | 6067 | 1.78 | 4366 | 5.05 |
| 11166 | 3.8 | 9465 | 3.77 | 7764 | 3.81 | 6063 | 1.78 | 4362 | 5.07 |
| 11162 | 3.78 | 9462 | 3.76 | 7761 | 3.8 | 6060 | 1.78 | 4359 | 5.26 |
| 11159 | 3.74 | 9458 | 3.76 | 7757 | 3.79 | 6056 | 1.79 | 4355 | 5.26 |
| 11155 | 3.68 | 9454 | 3.76 | 7753 | 3.79 | 6052 | 1.79 | 4351 | 5.05 |
| 11151 | 3.63 | 9450 | 3.76 | 7749 | 3.79 | 6048 | 1.79 | 4347 | 4.98 |
| 11147 | 3.62 | 9446 | 3.77 | 7745 | 3.78 | 6044 | 1.79 | 4343 | 5.11 |
| 11143 | 3.64 | 9442 | 3.78 | 7741 | 3.78 | 6040 | 1.8 | 4339 | 5.29 |
| 11139 | 3.66 | 9438 | 3.78 | 7737 | 3.78 | 6036 | 1.8 | 4335 | 5.32 |
| 11135 | 3.64 | 9435 | 3.77 | 7734 | 3.77 | 6033 | 1.81 | 4332 | 5.36 |
| 11132 | 3.61 | 9431 | 3.76 | 7730 | 3.76 | 6029 | 1.82 | 4328 | 5.4 |
| 11128 | 3.6 | 9427 | 3.76 | 7726 | 3.75 | 6025 | 1.82 | 4324 | 5.28 |
| 11124 | 3.64 | 9423 | 3.77 | 7722 | 3.74 | 6021 | 1.83 | 4320 | 5.13 |
| 11120 | 3.65 | 9419 | 3.78 | 7718 | 3.73 | 6017 | 1.84 | 4316 | 4.99 |
| 11116 | 3.63 | 9415 | 3.78 | 7714 | 3.72 | 6013 | 1.86 | 4312 | 4.95 |
| 11112 | 3.6 | 9411 | 3.8 | 7710 | 3.72 | 6009 | 1.87 | 4308 | 4.95 |
| 11108 | 3.56 | 9408 | 3.81 | 7707 | 3.72 | 6006 | 1.88 | 4305 | 4.95 |
| 11105 | 3.54 | 9404 | 3.82 | 7703 | 3.7 | 6002 | 1.89 | 4301 | 4.95 |
| 11101 | 3.53 | 9400 | 3.83 | 7699 | 3.68 | 5998 | 1.91 | 4297 | 4.97 |
| 11097 | 3.52 | 9396 | 3.84 | 7695 | 3.66 | 5994 | 1.93 | 4293 | 5.01 |
| 11093 | 3.5 | 9392 | 3.83 | 7691 | 3.65 | 5990 | 1.94 | 4289 | 5.06 |
| 11089 | 3.5 | 9388 | 3.83 | 7687 | 3.65 | 5986 | 1.96 | 4285 | 5.1 |
| 11085 | 3.51 | 9384 | 3.84 | 7683 | 3.65 | 5982 | 1.98 | 4281 | 5.17 |
| 11081 | 3.52 | 9381 | 3.86 | 7680 | 3.65 | 5979 | 2 | 4278 | 5.37 |
| 11078 | 3.54 | 9377 | 3.87 | 7676 | 3.64 | 5975 | 2.02 | 4274 | 5.28 |
| 11074 | 3.54 | 9373 | 3.88 | 7672 | 3.63 | 5971 | 2.04 | 4270 | 5.36 |
| 11070 | 3.51 | 9369 | 3.89 | 7668 | 3.63 | 5967 | 2.07 | 4266 | 5.34 |
| 11066 | 3.5 | 9365 | 3.91 | 7664 | 3.63 | 5963 | 2.09 | 4262 | 5.21 |
| 11062 | 3.52 | 9361 | 3.92 | 7660 | 3.62 | 5959 | 2.11 | 4258 | 5.13 |
| 11058 | 3.53 | 9357 | 3.91 | 7656 | 3.61 | 5955 | 2.14 | 4254 | 5.15 |
| 11054 | 3.53 | 9354 | 3.9 | 7653 | 3.6 | 5952 | 2.17 | 4251 | 5.16 |
| 11051 | 3.52 | 9350 | 3.89 | 7649 | 3.6 | 5948 | 2.2 | 4247 | 5.23 |
| 11047 | 3.52 | 9346 | 3.91 | 7645 | 3.59 | 5944 | 2.24 | 4243 | 5.21 |
| 11043 | 3.51 | 9342 | 3.94 | 7641 | 3.59 | 5940 | 2.28 | 4239 | 5.19 |
| 11039 | 3.5 | 9338 | 3.99 | 7637 | 3.57 | 5936 | 2.34 | 4235 | 5.27 |
| 11035 | 3.5 | 9334 | 4.02 | 7633 | 3.56 | 5932 | 2.4 | 4231 | 5.24 |
| 11031 | 3.52 | 9330 | 4.02 | 7629 | 3.56 | 5928 | 2.48 | 4227 | 5.09 |
| 11027 | 3.53 | 9327 | 4.01 | 7626 | 3.55 | 5925 | 2.58 | 4224 | 5.08 |
| 11024 | 3.52 | 9323 | 4.01 | 7622 | 3.54 | 5921 | 2.68 | 4220 | 5.19 |
| 11020 | 3.53 | 9319 | 4.04 | 7618 | 3.54 | 5917 | 2.79 | 4216 | 5.39 |
| 11016 | 3.52 | 9315 | 4.06 | 7614 | 3.53 | 5913 | 2.89 | 4212 | 5.46 |
| 11012 | 3.5 | 9311 | 4.05 | 7610 | 3.53 | 5909 | 2.98 | 4208 | 5.34 |
| 11008 | 3.49 | 9307 | 4.06 | 7606 | 3.53 | 5905 | 3.03 | 4204 | 5.23 |
| 11004 | 3.5 | 9303 | 4.09 | 7602 | 3.52 | 5901 | 3.06 | 4200 | 5.14 |
| 11000 | 3.52 | 9300 | 4.1 | 7599 | 3.51 | 5898 | 3.07 | 4197 | 5.13 |
| 10997 | 3.54 | 9296 | 4.1 | 7595 | 3.5 | 5894 | 3.09 | 4193 | 5.1 |
| 10993 | 3.53 | 9292 | 4.11 | 7591 | 3.5 | 5890 | 3.12 | 4189 | 5.02 |
| 10989 | 3.52 | 9288 | 4.11 | 7587 | 3.49 | 5886 | 3.17 | 4185 | 5.03 |
| 10985 | 3.51 | 9284 | 4.11 | 7583 | 3.48 | 5882 | 3.25 | 4181 | 5.07 |
| 10981 | 3.51 | 9280 | 4.1 | 7579 | 3.48 | 5878 | 3.34 | 4177 | 5.09 |
| 10977 | 3.53 | 9276 | 4.1 | 7575 | 3.48 | 5874 | 3.44 | 4173 | 5.09 |
| 10973 | 3.55 | 9273 | 4.14 | 7572 | 3.47 | 5871 | 3.52 | 4170 | 5.14 |
| 10970 | 3.56 | 9269 | 4.18 | 7568 | 3.46 | 5867 | 3.57 | 4166 | 5.22 |
| 10966 | 3.55 | 9265 | 4.17 | 7564 | 3.46 | 5863 | 3.58 | 4162 | 5.26 |
| 10962 | 3.53 | 9261 | 4.17 | 7560 | 3.45 | 5859 | 3.57 | 4158 | 5.27 |
| 10958 | 3.53 | 9257 | 4.17 | 7556 | 3.44 | 5855 | 3.55 | 4154 | 5.33 |
| 10954 | 3.56 | 9253 | 4.17 | 7552 | 3.44 | 5851 | 3.53 | 4150 | 5.35 |
| 10950 | 3.6 | 9249 | 4.18 | 7548 | 3.43 | 5847 | 3.52 | 4146 | 5.29 |
| 10946 | 3.6 | 9246 | 4.2 | 7545 | 3.42 | 5844 | 3.53 | 4143 | 5.25 |
| 10943 | 3.58 | 9242 | 4.19 | 7541 | 3.41 | 5840 | 3.54 | 4139 | 5.27 |
| 10939 | 3.57 | 9238 | 4.19 | 7537 | 3.41 | 5836 | 3.58 | 4135 | 5.34 |
| 10935 | 3.59 | 9234 | 4.24 | 7533 | 3.4 | 5832 | 3.63 | 4131 | 5.21 |
| 10931 | 3.59 | 9230 | 4.38 | 7529 | 3.4 | 5828 | 3.69 | 4127 | 5.14 |
| 10927 | 3.59 | 9226 | 4.44 | 7525 | 3.4 | 5824 | 3.75 | 4123 | 5.07 |
| 10923 | 3.56 | 9222 | 4.3 | 7521 | 3.39 | 5820 | 3.82 | 4119 | 4.99 |
| 10919 | 3.53 | 9219 | 4.2 | 7518 | 3.38 | 5817 | 3.89 | 4116 | 4.96 |
| 10916 | 3.51 | 9215 | 4.21 | 7514 | 3.37 | 5813 | 3.96 | 4112 | 5.02 |
| 10912 | 3.51 | 9211 | 4.27 | 7510 | 3.36 | 5809 | 4.02 | 4108 | 5.19 |
| 10908 | 3.51 | 9207 | 4.33 | 7506 | 3.36 | 5805 | 4.07 | 4104 | 5.42 |
| 10904 | 3.52 | 9203 | 4.37 | 7502 | 3.35 | 5801 | 4.1 | 4100 | 5.49 |
| 10900 | 3.53 | 9199 | 4.35 | 7498 | 3.35 | 5797 | 4.11 | 4096 | 5.37 |
| 10896 | 3.54 | 9195 | 4.31 | 7494 | 3.34 | 5793 | 4.09 | 4092 | 5.28 |
| 10892 | 3.57 | 9192 | 4.3 | 7491 | 3.33 | 5790 | 4.07 | 4089 | 5.2 |
| 10889 | 3.59 | 9188 | 4.29 | 7487 | 3.33 | 5786 | 4.04 | 4085 | 5.09 |
| 10885 | 3.57 | 9184 | 4.3 | 7483 | 3.32 | 5782 | 3.99 | 4081 | 5.01 |
| 10881 | 3.56 | 9180 | 4.32 | 7479 | 3.32 | 5778 | 3.93 | 4077 | 4.97 |
| 10877 | 3.58 | 9176 | 4.32 | 7475 | 3.31 | 5774 | 3.86 | 4073 | 4.96 |
| 10873 | 3.61 | 9172 | 4.28 | 7471 | 3.31 | 5770 | 3.76 | 4069 | 4.99 |
| 10869 | 3.62 | 9168 | 4.22 | 7467 | 3.3 | 5766 | 3.66 | 4065 | 5.02 |
| 10865 | 3.62 | 9165 | 4.17 | 7464 | 3.29 | 5763 | 3.55 | 4062 | 5.01 |
| 10862 | 3.64 | 9161 | 4.16 | 7460 | 3.29 | 5759 | 3.46 | 4058 | 5.15 |
| 10858 | 3.64 | 9157 | 4.19 | 7456 | 3.28 | 5755 | 3.37 | 4054 | 5.36 |
| 10854 | 3.63 | 9153 | 4.23 | 7452 | 3.28 | 5751 | 3.28 | 4050 | 5.4 |
| 10850 | 3.62 | 9149 | 4.27 | 7448 | 3.27 | 5747 | 3.21 | 4046 | 5.31 |
| 10846 | 3.64 | 9145 | 4.34 | 7444 | 3.27 | 5743 | 3.14 | 4042 | 5.2 |
| 10842 | 3.68 | 9141 | 4.41 | 7440 | 3.26 | 5739 | 3.09 | 4038 | 5.14 |
| 10838 | 3.71 | 9138 | 4.43 | 7437 | 3.26 | 5736 | 3.05 | 4035 | 5.05 |
| 10835 | 3.7 | 9134 | 4.4 | 7433 | 3.25 | 5732 | 3.03 | 4031 | 4.95 |
| 10831 | 3.66 | 9130 | 4.39 | 7429 | 3.25 | 5728 | 3.02 | 4027 | 4.93 |
| 10827 | 3.64 | 9126 | 4.37 | 7425 | 3.24 | 5724 | 3.02 | 4023 | 5.05 |
| 10823 | 3.64 | 9122 | 4.29 | 7421 | 3.24 | 5720 | 3.03 | 4019 | 5.23 |
| 10819 | 3.65 | 9118 | 4.27 | 7417 | 3.23 | 5716 | 3.04 | 4015 | 5.32 |
| 10815 | 3.67 | 9114 | 4.31 | 7413 | 3.23 | 5712 | 3.05 | 4011 | 5.37 |
| 10811 | 3.68 | 9111 | 4.34 | 7410 | 3.22 | 5709 | 3.06 | 4008 | 5.14 |
| 10808 | 3.66 | 9107 | 4.34 | 7406 | 3.22 | 5705 | 3.08 | 4004 | 4.88 |
| 10804 | 3.63 | 9103 | 4.37 | 7402 | 3.21 | 5701 | 3.1 | 4000 | 4.81 |
| 10800 | 3.62 | 9099 | 4.43 | 7398 | 3.21 | 5697 | 3.12 | | |
| 10796 | 3.62 | 9095 | 4.44 | 7394 | 3.21 | 5693 | 3.15 | | |

1. A system for evaluating a crude oil sample and calculating indicative properties of a gas oil fraction of the crude oil without first distilling the gas oil fraction from the crude oil, the system comprising:
a near infrared spectrometer;
a non-volatile memory device that stores calculation modules and data;
a processor coupled to the non-volatile memory;
a first calculation module that calculates a near infrared absorbance index from a summation of near infrared spectroscopy absorbance values of the crude oil for peaks detected at a predetermined series of wavenumbers;
a second calculation module that derives the cetane number for the gas oil fraction as a function of the near infrared absorbance index and density of the sample;
a third calculation module that derives the pour point for the gas oil fraction as a function of the near infrared absorbance index and density of the sample;
a fourth calculation module that derives the cloud point for the gas oil fraction as a function of the near infrared absorbance index and density of the sample; and
a fifth calculation module that derives the aniline point for the gas oil fraction as a function of the near infrared absorbance index and density of the sample.

2. The system of claim 1, wherein the gas oil boils in the nominal range 180-370° C.

3. The system of claim 1, wherein the wavenumber of near infrared spectrum is in the range 4,000-12,821 cm⁻¹.

4. A method for evaluating a crude oil sample and calculating indicative properties, the method comprising:
determining the density of the crude oil sample;
subjecting said crude oil sample to near infrared spectroscopy analysis;
calculating a near infrared absorbance index from a summation of near infrared spectroscopy spectra data absorbance values of the crude oil sample for peaks detected at a predetermined series of wavenumbers;
calculating a cetane number as a function of the near infrared absorbance and density of the sample;
calculating pour point as a function of the near infrared absorbance and density of the sample;
calculating a cloud point as a function of the near infrared absorbance and density of the sample; and
calculating an aniline point as a function of the near infrared absorbance and the density of the sample.

5. The method of claim 4, wherein the gas oil boils in the nominal range 180-370° C.

6. The method of claim 4, wherein the wavenumber of near infrared spectrum is in the range 4,000-12,821 cm⁻¹.

7. A system for evaluating a crude oil sample and determining an indicative property, the system comprising:
a near infrared spectrometer;
a non-volatile memory device that stores calculation modules and data, the data including density of the crude oil sample and near infrared spectroscopy absorbance values obtained from the near infrared spectrometer;
a processor coupled to the non-volatile memory;
a first calculation module that calculates a near infrared absorbance index from a summation of near infrared spectroscopy absorbance values of the crude oil for peaks detected at a predetermined series of wavenumbers; and
a second calculation module that calculates the indicative property as a function of the near infrared absorbance index and the density of the sample.

8. The system of claim 7, wherein the indicative property is the cetane number.

9. The system of claim 7, wherein the indicative property is the pour point.

10. The system of claim 7, wherein the indicative property is the cloud point.

11. The system of claim 7, wherein the indicative property is the aniline point.

12. A method for evaluating a crude oil sample and calculating an indicative property, the method comprising:
determining the density of the crude oil sample;
subjecting the crude oil sample to near infrared spectroscopy analysis;
calculating a near infrared absorbance index from a summation of near infrared spectroscopy spectra absorbance values of the crude oil sample for peaks detected at a predetermined series of wavenumbers; and
calculating and recording the indicative property as a function of the near infrared absorbance index and the density of the sample.

13. The method of claim 12, wherein the indicative property is the cetane number.

14. The method of claim 12, wherein the indicative property is the pour point.

15. The method of claim 12, wherein the indicative property is the cloud point.

16. The method of claim 12, wherein the indicative property is the aniline point.
